(19) United States
(12) Patent Application Publication
(10) Pub. No.: US 2013/0226089 A1
(43) Pub. Date: Aug. 29, 2013

(54) Medicated Module for a Drug Delivery Device

(71) Applicants: James Alexander Davies, Warwickshire (GB); Steven Wimpenny, Warwickshire (GB)

(72) Inventors: James Alexander Davies, Warwickshire (GB); Steven Wimpenny, Warwickshire (GB)

(73) Assignee: Sanofi-Aventis Deutschland GmbH, Frankfurt am Main (DE)

(21) Appl. No.: 13/882,708

(22) PCT Filed: Oct. 31, 2011
(86) PCT No.: PCT/EP11/69097

§ 371 Date: Apr. 30, 2013

Related U.S. Application Data
(60) Provisional application No. 61/432,707, filed on Jan. 14, 2011.

(30) Foreign Application Priority Data
Nov. 3, 2010 (EP) 10189779.1

(51) Int. Cl. A61M 5/315

(52) U.S. Cl. CPC A61M 5/31596
USPC 604/125; 604/205

(57) ABSTRACT

A method and system for sequentially dispensing a non-user settable dose of one medicament followed by a user-settable dose of a primary medicament. The system may include a dual cross-section, floating piston arranged to facilitate first delivering all or substantially all of a given medicament (e.g., a secondary medicament) through an output needle and then thereafter delivering yet another medicament (e.g., a primary medicament) through the same output needle in a single injection step. The dual cross-section, floating piston of the system also reduces or limits ullage left in the medicated module relative to the amount of the secondary medicament dispensed from the system.

### CROSS REFERENCE TO RELATED APPLICATIONS

The present application is a U.S. National Phase Application pursuant to 35 U.S.C. §371 of International Application No. PCT/EP2011/069097 filed Oct. 31, 2011, which claims priority to European Patent Application No. 10189779.1 filed Nov. 3, 2010 and U.S. Provisional Patent Application No. 61/432,707 filed Jan. 14, 2011. The entire disclosure contents of these applications are herewith incorporated by reference into the present application.

### FIELD OF INVENTION

This present patent application relates to medical devices and methods of delivering at least two drug agents from separate reservoirs using devices having only a single dose setting mechanism and a single dispense interface. A single delivery procedure initiated by the user causes a non-user settable dose of a second drug agent and a variable set dose of a first drug agent to be delivered to the patient. The drug agents may be available in two or more reservoirs, containers or packages, each containing independent (single drug compound) or pre-mixed (co-formulated multiple drug compounds) drug agents. Specifically, this application concerns methods and systems for sequentially dispensing a non-user settable dose of one medicament followed by a user-settable dose of a primary medicament. The disclosed methods and systems limit or minimize the ullage left in the medicated module after the dispensing process.

### BACKGROUND

Certain disease states require treatment using one or more different medicaments. Some drug compounds need to be delivered in a specific relationship with each other in order to deliver the optimum therapeutic dose. The presently proposed devices and methods are of particular benefit where combination therapy is desirable, but not possible in a single formulation for reasons such as, but not limited to, stability, compromised therapeutic performance and toxicology.

For example, in some cases it might be beneficial to treat a diabetic with a long acting insulin and with a glucagon-like peptide-1 (GLP-1), which is derived from the transcription product of the proglucagon gene. GLP-1 is found in the body and is secreted by the intestinal L cell as a gut hormone. GLP-1 possesses several physiological properties that make it (and its analogs) a subject of intensive investigation as a potential treatment of diabetes mellitus.

There are a number of potential problems when delivering two active medicaments or “agents” simultaneously. The two active agents may interact with each other during the long-term, shelf life storage of the formulation. Therefore, it is advantageous to store the active components separately and combine them at the point of delivery, e.g. injection, needle-less injection, pumps, or inhalation. However, the process for combining the two agents needs to be simple and convenient for the user to perform reliably, repeatedly and safely.

A further problem is that the quantities and/or proportions of each active agent making up the combination therapy may need to be varied for each user or at different stages of their therapy. For example one or more active agents may require a titration period to gradually introduce a patient up to a “maintenance” dose. A further example would be if one active agent requires a non-adjustable fixed dose while the other is varied in response to a patient's symptoms or physical condition. This problem means that pre-mixed formulations of multiple active agents may not be suitable as these pre-mixed formulations would have a fixed ratio of the active components, which could not be varied by the healthcare professional or user.

Additional problems arise where a multi-drug compound therapy is required, because many users cannot cope with having to use more that one drug delivery system or make the necessary accurate calculation of the required dose combination. This is especially true for users with dexterity or computational difficulties. Further, for some drug combinations for which this delivery of two medicaments in a single injection step is desirable, it may be additionally desirable for the two medicaments to be delivered sequentially (i.e., one after the other, with minimal or no opportunity for mixing).

In addition, in some circumstances, it may be necessary to offset a dialed dose (i.e., what is indicated to the user) to accommodate the effect of ullage in the device after dispense. Reducing the degree to which the dialled dose may need to be offset in such circumstances is desirable. Further, limiting or reducing the amount of medicament that is wasted by being left in the device after dispense may also be desirable.

Accordingly, there exists a need to provide devices and methods for the sequential delivery of two or more medicaments in a single injection or delivery step that is simple for the user to perform and that limits the ullage in the device after dispense.

The presently proposed devices and methods overcome the above-mentioned problems by providing separate storage containers for two or more active drug agents. A drug delivery device comprising a first container and a medicated module comprising a second container. The drug agents are then delivered sequentially to the patient during a single delivery procedure. Beneficially, delivering the drugs sequentially may avoid or limit mixing of the drug agents. Setting a dose of one medicament automatically fixes or determines the dose of the second medicament (i.e., non-user settable). Further, utilizing dual cross-section configurations of the piston for the proposed medicated module beneficially facilitates limiting the ullage left in the medicated module after dispense. Specifically, the amount of primary medicament left in the medicated module after dispense is less than the volume of the second medicament in the medicated module originally present prior to dispense. This dual cross-section piston configuration also beneficially reduces the degree to which the dialed dose on the drug delivery device may need to be offset to accommodate the affect of ullage in the medicated module. The piston may be so called “floating piston”; that is, the piston is not connected to a piston rod and is free to move axially (e.g. in the body of the medicated module) when an axially-directed force is applied. It should be understood that although the piston is referred to as a floating piston, the piston is securely held in the body.

The proposed devices and methods also give the opportunity for varying the quantity of one or both medicaments. For example, one fluid quantity can be varied by changing the properties of the injection device (e.g., dialing a user variable dose or changing the device's “fixed” dose). The second fluid quantity can be changed by manufacturing a variety of secondary drug containing packages with each variant containing a different volume and/or concentration of the second active agent. The user or healthcare professional would then select the most appropriate secondary package or series or combination of series of different packages for a particular treatment regime. The proposed medicated module contains a self-contained reservoir in which non-user-settable dose of a medicament may be stored.

These and other advantages will become evident from the following more detailed description of the invention.

### SUMMARY

The presently proposed devices and methods allow for complex combinations of multiple drug compounds within a single drug delivery system. Further, the presently proposed devices and methods allow the user to set and sequentially dispense at least two drug agents through one single dose setting mechanism and a single dispense interface. Still further, the presently proposed devices and methods limit or reduce the amount of ullage in the drug delivery system after the dispense process. This single dose setter controls the mechanism of the device such that a predefined combination of the individual drug compounds is delivered when a single dose of one of the medicaments is set and dispensed through the single dispense interface.

By defining the therapeutic relationship between the individual drug compounds, the proposed delivery device and delivery methods help ensure that a patient/user receives the optimum therapeutic combination dose from a multi-drug compound device without the inherent risks associated with multiple inputs where the user has to calculate and set the correct dose combination every time they use the device. The medicaments can be fluids, defined herein as liquids or gases or powders that are capable of flowing and that change shape at a steady rate when acted upon by a force tending to change its shape. Alternatively, one or both of the medicaments may be a solid that is carried, solubilized or otherwise dispensed with another fluid medicament.

Applicants' proposed concept is of particular benefit to users with dexterity or computational difficulties as the single input and associated predefined therapeutic profile removes the need for them to calculate their prescribed dose every time they use the device and the single input allows considerably easier setting and dispensing of the combined compounds.

In a preferred embodiment a master drug compound, such as insulin, contained within a multiple dose, user selectable drug delivery device could be used with a single use, user replaceable, medicated module that contains a single dose of a secondary medicament and the single dispense interface. When connected to the primary drug delivery device, the secondary compound is activated/delivered on dispense of the primary compound. Although the present application specifically mentions insulin, insulin analogs or insulin derivatives, and GLP-1 or GLP-1 analogs as two possible drug combinations, other drugs or drug combinations, such as an analgesics, hormones, beta agonists or corticosteroids, or a combination of any of the above-mentioned drugs could be used with our proposed method and system.

For the purposes of our proposed method and system the term “insulin” shall mean Insulin, insulin analogs, insulin derivatives or mixtures thereof, including human insulin or a human insulin analogs or derivatives. Examples of insulin analogs are, without limitation, Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin or Des(B30) human insulin. Examples of insulin derivatives are, without limitation, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(w-carboxyheptadecanoyl)-des(B30) human insulin and B29-N-(ω-carboxyhepta [symbol] decanoyl) human insulin.

As used herein the term “GLP-1” shall mean GLP-1, GLP-1 analogs, or mixtures thereof, including without limitation, exenatide (Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2), Exendin-3, Liraglutide, or AVE0010 (H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-Lys-Lys-Lys-NH2).

Examples of beta agonists are, without limitation, salbutamol, levosalbutamol, terbutaline, pirbuterol, procaterol, metaproterenol, fenoterol, bitolterol mesylate, salmeterol, formoterol, bambuterol, clenbuterol, indacaterol.

Hormones are for example hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

According to an embodiment, a medicated module is attachable to a drug delivery device that includes a drug reservoir holding a first medicament. The medicated module includes a first needle, a second needle, a piston, a retention feature, and a second medicament. The piston has a first portion and a second portion, and an area of a cross-section of the first portion is smaller than an area of a cross-section of the second portion. The retention feature has an indentation, and at least part of the first portion of the piston is located in the indentation. Further, the second portion of the piston is located on a distal side of the retention feature. The second medicament is located on a distal side of the piston in a cavity defined by a distal surface of the medicated module and a distal end of the second portion of the piston. After the medicated module is attached to the drug delivery device, (i) the first needle is in fluid communication with the drug reservoir and (ii) the piston prevents fluid communication between the first and second needle. During dispensing, the first medicament flows through the first needle into a second cavity formed by the retention feature and the first portion of the piston, and the first medicament forces the piston to move in a distal direction toward the distal surface. The second portion of the piston forces substantially all of the second medicament to flow through the second needle. After substantially all of the second medicament is forced to flow through the second needle, the second needle fully pierces the piston, thereby opening up fluid communication between the first needle and the second needle.

According to another embodiment, a similar medicated module is provided where the second medicament is disposed in a flexible membrane. According to yet another embodiment, a medicated module is attachable to a drug delivery device that has a drug reservoir holding a first medicament. The medicated module includes an output needle, a floating piston disposed in the output needle, and a piercing needle having a piercing tip securely fixed in the output needle. Dispense of the first medicament forces the floating piston to move in a distal direction, and this distal movement forces the second medicament out of the output needle. At a predefined amount of movement in the distal direction, the piercing tip fully pierces the floating piston, thereby opening up fluid communication between the first medicament and an output of the output needle.

A medicated module in accordance with Applicants' proposed concept can be designed for use with any drug delivery device with an appropriate compatible interface. However, it may be preferable to design the module in such a way as to limit its use to one exclusive primary drug delivery device (or family of devices) through employment of dedicated or coded features to prevent attachment of a non-appropriate medicated module to a non-matching device. In some situations it may be beneficial to ensure that the medicated module is exclusive to one drug delivery device while also permitting the attachment of a standard drug dispense interface to the device. This would allow the user to deliver a combined therapy when the module is attached, but would also allow delivery of the primary compound independently through a standard drug dispense interface in situations, such as, but not limited to, dose splitting or top-up of the primary compound.

A particular benefit of Applicants' method and system is that the method and system allow for sequential dosing of a first medicament and a second medicament through a single dispense interface. Thus, the method and system beneficially prevents or limits mixing of the first, primary medicament and the second medicament. This may be beneficial, for example, when mixing of medicaments prior to delivery negatively or detrimentally affects at least one of the medicaments. Another particular benefit of Applicants' method and system is that the method and system reduces or limits the ullage left in the medicated module after dispense. Specifically, the ullage is reduced relative to the volume of the secondary medicament that is dispensed from the medicated module.

In a preferred embodiment, the primary drug delivery device is used more than once and therefore is a multi-use device; however, the drug delivery device may also be a single use disposable device. Such a device may or may not have a replaceable reservoir of the primary drug compound, but our proposed concept is equally applicable to both scenarios. It is also possible to have a suite of different medicated modules for various conditions that could be prescribed as one-off extra medication to patients already using a standard drug delivery device. Should the patient attempt to reuse a previously used medicated module, features may be present that prevent reattachment to a primary drug delivery device or that prevent or discourage subsequent dosing through the needle via alternative means. For example, this module may include a locking needle guard that is activated after a user delivers a dose from the medicated module. Other means of alerting the user may include some (or all) of the following:

Physical prevention of medicated module re-attachment to the primary drug deliver device once the module has been used and removed.

Physical/hydraulic prevention of subsequent liquid flow through the drug dispense interface once it has been used.

Physical locking of the dose setter and/or dose button of the primary drug delivery device.

Visual warnings (e.g., change in color and/or warning text/indicia within an indication window on the module once insertion and/or fluid flow has occurred).

Tactile feedback (presence or absence of tactile features on the outer surface of the module hub following use).

A further proposed feature is that both medicaments are delivered via one injection needle and in one injection step. This offers a convenient benefit to the user in terms of reduced user steps compared to administering two separate injections. This convenience benefit may also result in improved compliance with the prescribed therapy, particularly for users who find injections unpleasant or who have computational or dexterity difficulties.

These as well as other advantages of various aspects of the present invention will become apparent to those of ordinary skill in the art by reading the following detailed description, with appropriate reference to the accompanying drawings.

### BRIEF DESCRIPTION OF THE DRAWINGS

Exemplary embodiments are described herein with reference to the drawings, in which:

FIG. 1 illustrates a perspective view of one possible drug delivery device that can be used with Applicants' proposed medicated module;

FIG. 2a illustrates a cross-sectional view of an exemplary medicated module attached to an exemplary drug delivery device;

FIG. 2b illustrates a detailed cross-sectional view of the exemplary medicated module of FIG. 2a;

FIG. 3 illustrates a cross-sectional view of the exemplary medicated module and exemplary drug delivery device of FIG. 2 after the piston is fully pierced;

FIG. 4a illustrates a cross-sectional view of an exemplary medicated module attached to an exemplary drug delivery device;

FIG. 4b illustrates a detailed cross-sectional view of the exemplary medicated module of FIG. 4a;

FIG. 5 illustrates a partial cross-sectional view of an exemplary medicated module; and

FIG. 6 illustrates a partial cross-sectional view of the exemplary medicated module of FIG. 5 after a needle fully pierces the floating piston of the medicated module.

### DETAILED DESCRIPTION

Applicants' proposed system and method relates to sequentially dispensing a non-user settable dose of one medicament followed by a user-settable dose of a primary medicament using a single dispense interface. The proposed system and methods relate specifically to a method and system that utilizes a dual cross-section piston that facilitates first delivering all or substantially all of a given medicament (e.g., a secondary medicament) through an output needle and then thereafter delivering yet another medicament (e.g., a primary medicament) through the same output needle in a single injection step. The dual cross-section piston also beneficially reduces or limits ullage left in the medicated module relative to the amount of the given, secondary medicament dispensed from the medicated module.

A medicated module in accordance with proposed embodiments may be attached to a primary drug delivery device, such as drug delivery device 100. Generally, in given embodiments, Applicants' proposed medicated module includes a dual cross-section, floating piston that can facilitate (i) sequential dosing of a secondary medicament followed by a primary medicament and (ii) limiting the ullage left in the medicated module.

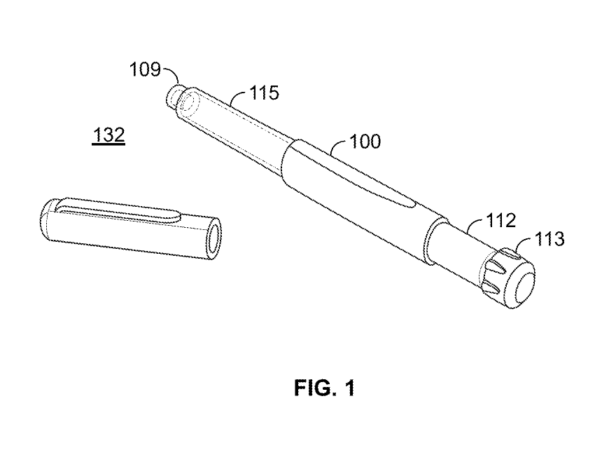

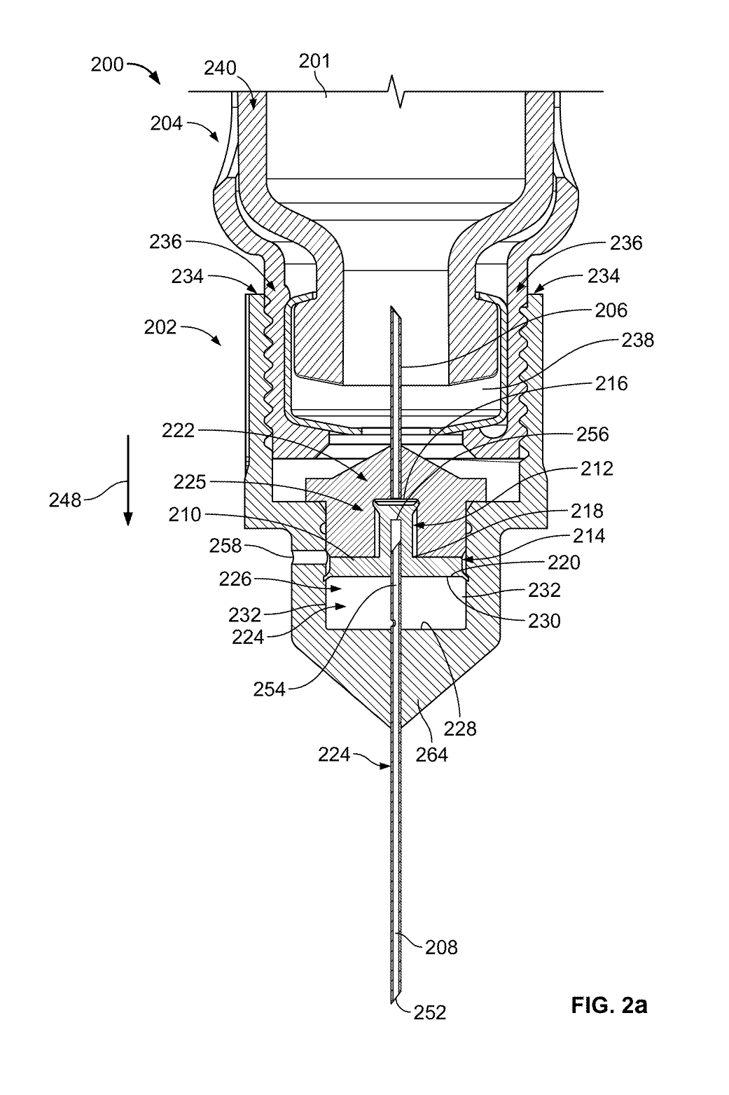

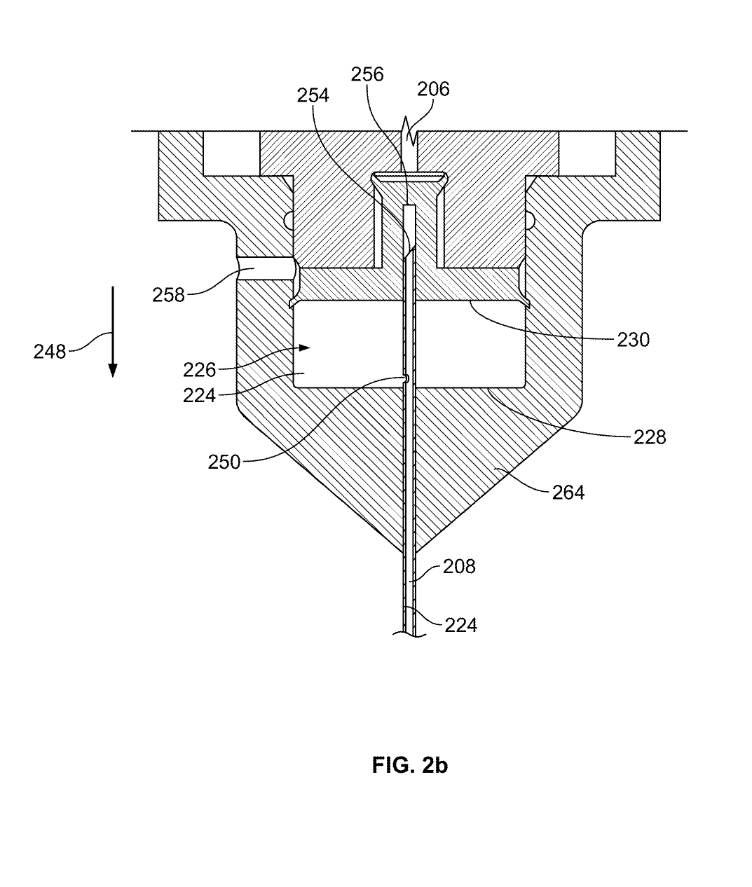

FIG. 1 illustrates one example of a drug delivery device 100 that a medicated module, such as the exemplary medicated modules depicted in FIGS. 2-6, can be attached to. Specifically, the medicated module can be attached to the connection means 109 of distal end 132. A medicated module in accordance with Applicants' proposed concept is preferably self-contained and may be provided as a sealed and sterile disposable module that has an attachment means compatible to the attachment means 109 at the distal end 132 of device 100. Although not shown, the medicated module could be supplied by a manufacturer contained in a protective and sterile container, where the user would peel or rip open a seal or the container itself to gain access to the sterile medicated module. Further, the drug delivery device 100 includes a housing including a single dose setter 112. The dose setter 112 may be operably connected to a primary reservoir of medicament that may be stored in the drug delivery device, such as in cartridge holder 115. The user may use a dose dial button 113 in order to dial a user selectable dose of the primary medicament.

Applicants' proposed medicated module is attachable to a drug delivery (such as drug delivery device 100) that has a drug reservoir holding a first, primary medicament. In a first embodiment, the medicated module includes a first needle, a second needle, and a dual cross-section piston. The dual cross-section piston has a first portion and a second portion, and an area of a cross-section of the first portion is smaller than an area of a cross-section of the second portion. Preferably, the area of the cross-section of the first portion is substantially smaller (e.g., 2-10 times smaller) than the area of the cross-section of the second portion. Further, the medicated module includes a retention feature having an indentation. At least part of the first portion of the piston is located in the indentation, and the second portion of the piston is located on a distal side of the indentation feature. Preferably, prior to use, all of or substantially all of the first portion is located in the indentation of the retention feature.

Still further, the medicated module comprises a second medicament located on a distal side of the piston in a cavity defined by a distal surface of the medicated module and a distal end of the second portion of the piston. After the medicated module is attached to the drug delivery device, (i) the first needle is in fluid communication with the drug reservoir and (ii) the piston prevents fluid communication between the first and second needle. During dispensing, the first medicament flows through the first needle into a second cavity formed by the retention feature and the first portion of the piston. The force of the first medicament forces the piston to move in a distal direction toward the distal surface, and the second portion of the piston forces substantially all of the second medicament to flow through the second needle. After substantially all of the second medicament is forced to flow through the second needle, the second needle fully pierces the piston, thereby opening up fluid communication between the first needle and the second needle. The primary medicament may then be dispensed through the second needle. Beneficially, the dual cross-section arrangement of the floating piston limits (relative to the volume of the second medicament dispensed from the medicated module) the ullage left in the medicated module after the first and second medicaments are dispensed.

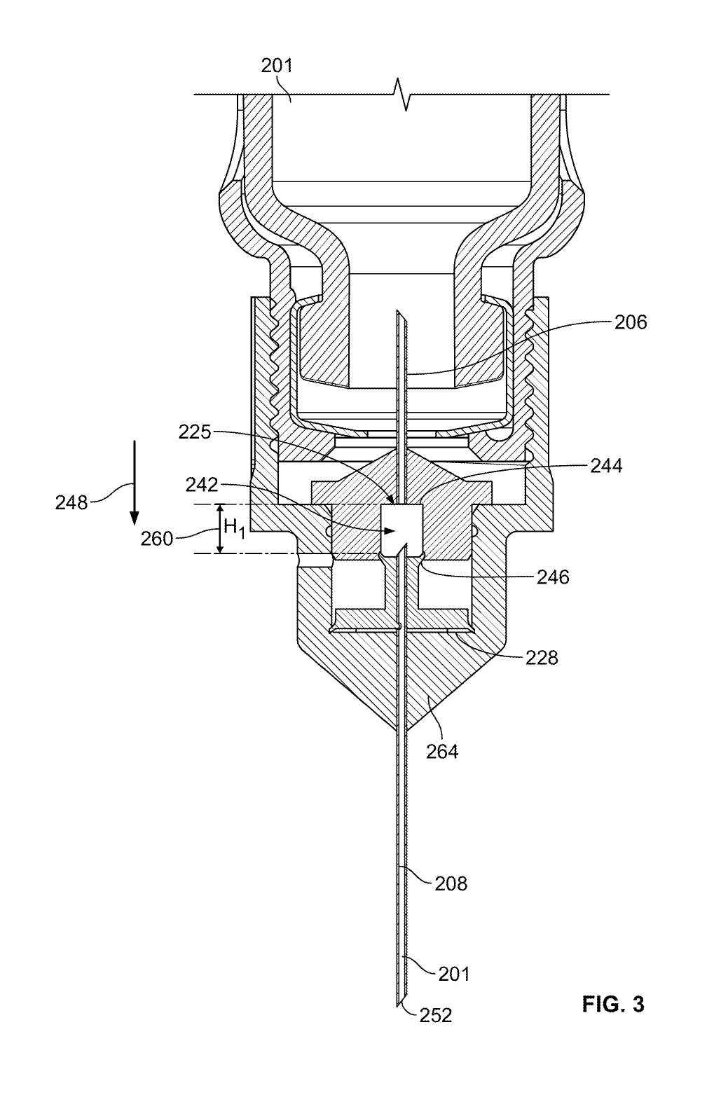

The first embodiment is now described with reference to FIGS. 2a-b and FIG. 3. Specifically, FIG. 2a illustrates a drug delivery system 200 including medicated module 202 and primary drug delivery device 204 that contains a first, primary medicament 201. In FIGS. 2a and 3, only a partial view of the distal end of the primary drug delivery device 204 is shown. Primary drug delivery device 204 may be the same as or similar to drug delivery device 100 of FIG. 1. The medicated module 202 includes a first needle 206 and a second needle 208. The first needle 206 may be referred to herein as an “engagement needle”, as the needle engages with or communicates with the reservoir of drug delivery device 204 when the module 202 and device 204 are attached. Further, the second needle 208 may be referred to herein as an “output needle”, as the second needle may be used to subcutaneously inject medicament into an injection site, such as an injection site of a user of drug delivery system 200.

The medicated module 202 also includes a piston 210. The piston 210 is a dual cross-section piston. That is, the cross-sections of the piston 210 are not consistent along the height of the piston, such that the piston includes at least two different cross-sectional sealing regions. Specifically, the piston 210 has a first portion 212 and a second portion 214. The first portion extends from axial point 216 to axial point 218, and the second portion 214 extends from axial point 218 to axial point 220.

As is evident from the Figures, the area of a cross-section of the first portion is less than the area of a cross-section of the second portion 214. The area of the cross-section of the first portion is preferably substantially less than the area of the cross-section of the second portion. In a preferred embodiment, the area of the cross-section of the second portion is at least double the area of the cross-section of the first portion. For example, the area of the cross-section of the second portion may be 2-25 times greater than the area of the cross-section of the first portion. In this example, the radius of the second portion 214 is approximately three times the radius of the first portion 212. Thus, since the area of a circle is A=πr2, the area of the cross-section of the second portion is approximately nine times the area of the cross-section of the first portion. As will be described in greater detail later, this dual cross-section arrangement of the piston 210 facilitates limiting the ullage in the module after dispense relative to the volume of the secondary medicament dispensed.

Returning to FIGS. 2a and 2b, the medicated module 202 also contains a medicament retention feature 222. Retention feature 222 comprises an indentation 225. Medicament retention feature 222 is contained in the body of the medicated module and is shaped to conform to the proximal end of piston 210. This retention feature can be a separate part or integral with the medicated module housing. As mentioned above, at least part of the first portion 212 is located in the indentation 225, and the second portion 214 is located on a distal side of the retention feature 222. Preferably, before the dosing process begins, the entire first portion 212 is located in the indentation 225, as depicted. However, in some examples, prior to dosing, part of the first portion may extend beyond the indentation. Further, in some examples, the height of the indentation may be greater than the height of the first portion, although this is not preferred.

Medicated module 202 also includes a second medicament 224. The second medicament is located on a distal side of the piston 210 in a cavity 226 that is defined by a distal surface such as bottom surface 228 of the medicated module 202, a distal end 230 of the second portion 214 of the piston 210, and sidewalls 232 of the medicated module. Some of the second medicament 224 may also be located in the second needle 208, as shown, which would be sealed off toward it's distal end (not shown). For the purposes of this disclosure, distal surface indicates an internal surface of the medicated module that is located on the distal end of the module and below the floating piston 210. Bottom surface 228 is an example. However, it should be understood that the distal surface may be located above true bottom surface of the module.

The medicated module 202 also includes an attachment means 234. The attachment means 234 is configured to attach to a corresponding attachment means of a drug delivery device, such as the attachment means 236 of drug delivery device 204 (or, in another example, the attachment means 109 at the distal end 132 of device 100). Further, in a preferred embodiment, the medicated module 202 also includes a needle cover or needle guard (not shown). A needle cover may have a connection feature (e.g., a snap-fit feature) that allows the cover to be removably attached to the body of the medicated module 202 and may additionally include a pierceable or removable sealing feature to completely enclose and encapsulate the secondary medicament prior to use. A needle cover or needle guard may substantially conceal the second needle 208 from a user's view so as to beneficially reduce any needle anxiety that a patient may be experiencing. While substantially concealing the needle, the needle cover or needle guard also helps to prevent inadvertent needle sticks.

FIG. 2a depicts the medicated module 202 after the module is attached to drug delivery device 204 and prior to the dispense process. Attachment of medicated module 202 to drug delivery device 204 causes the engagement needle 206 to penetrate the septum 238 of the drug cartridge or reservoir 240 of the drug delivery device 204. Once the engagement needle 206 has passed through the septum 238 of the cartridge 240, fluid connection is made with the first, primary medicament 201. In other words, the first needle 206 is in fluid communication with the drug reservoir 240. At this stage, the piston 210 separates the first needle 206 and second needle 208, thereby preventing fluid communication between the two needles 206, 208. Thus, the first medicament 201 does not interact with (e.g., mix with) second medicament 224.

After the module 202 is attached to the device 204, a user may set a user-settable dose of the first medicament 201. The dose of the drug delivery device may be set in a usual manner (e.g., by dialing out an appropriate number of units of the primary medicament 201 with a dose dial). Dispense of the second medicament 224 followed by the first, primary medicament 201 may then be achieved via activation of the dosing mechanism of the drug delivery device. Dispense of the medicaments 224, 201 is described with reference to FIGS. 2a, 2b, and 3.

Referring to FIG. 3, as the primary medicament 201 is dispensed from the reservoir 240, the medicament 201 flows through the engagement needle 206 into a cavity 242 that is formed between the proximal end 244 of the indentation 225 and outer proximal end surface 246 of the piston 210. It should be understood that the size of the cavity 242 is dynamic. That is, the cavity 242 changes size (i.e., height) throughout the dosing process. Specifically, the cavity 242 changes from an initial relatively very small height to a height of H1 260, which corresponds to the amount of distal travel of the piston 210. Additionally, the size of cavity 226 is similarly dynamic (the height of cavity 226 decreases as the height of cavity 242 increases). Retention feature 222 may also be referred to herein as a “retention cap”, as the retention feature 222 forms a “cap” over the piston 210, retaining the medicament 201 in the cavity 242.

The retention cap 222 is preferably substantially rigid. As an example, the retention cap may be composed of substantially rigid materials, such as an engineering polymer (e.g. Polypropylene—PP, Acrylonitrile Butadiene Styrene—ABS, Cyclo Olefin Polymer—COP, Polyethylene terephthalate—PET, Polycarbonate—PC, Polyoxymethylene—POM, Low Density Polyethylene—LDPE and others), depending, in part, on the drug compatibility/stability properties required. Since the retention cap 222 is substantially rigid and the first medicament 201 is generally or effectively incompressible, the piston 210 is displaced axially in distal direction 248 as the first medicament 201 is dispensed into cavity 242. As mentioned above, the retention cap 222 is integral with or a separate part fixed in the medicated module 202. Thus, the retention cap 222 will not be moved when the first medicament 201 is forced into the dynamically-sized cavity 242.

In the example of FIGS. 2a-b and 3, the piston 210 is preferably a “floating piston”. That is, the piston is not connected to a piston rod and is free to move axially in the body of the medicated module upon an axially-directed force. It should be understood that although the piston 210 is referred to as a floating piston, the piston 210 is securely held in the body. Specifically, the floating piston is in sliding and substantially fluid-tight engagement with the medicated module. Specifically, the first portion 212 is in sliding and substantially fluid tight engagement with an inner wall of the indentation 225 on the retention cap 222. Preferably, the proximal end of the piston is where the sealing interface is located and the remainder of portion 212 is in clearance with the inner wall of indentation 225. Further, the second portion 214 is in sliding and substantially fluid tight engagement with inner sidewalls 232 of the medicated module 202. Preferably, the distal end of the piston is where the sealing interface is located and the remainder of portion 214 is in clearance with the inner sidewalls 232. Therefore, the second portion 214 does not allow the second medicament 224 to travel in the proximal direction above the second portion 214. Similarly, the first portion 212 does not allow the primary medicament 201 to flow or travel in the distal direction beyond the first portion 212.

The fluid-tight engagement, however, still allows for the piston 210 to slide axially upon the force of primary medicament 201 flowing into cavity 242 when the medicament is dispensed by a user. As seen when FIGS. 2a and 2b are compared to FIG. 3, dispense of the first medicament 201 forces the piston 210 to move in the distal direction 248. This distal movement forces the second medicament 224 out of the second needle. Specifically, the movement of the piston 210 causes the second medicament 224 to flow through a side hole 250 located in the output needle 208. The axis of the side hole 250 is preferably perpendicular to the axis of the output needle and the side hole 250 is preferably located near the bottom surface 228 of the medical module 202 (e.g., within 0.5-1 millimeter (mm)) so as to minimize the amount of the secondary medicament that remains in this region after use. The secondary medicament 224 is then forced through the needle 208 and out of output 252 at the distal end of the needle 208.

At a predetermined amount of distal travel of the piston 210, the piercing tip 254, located at the proximal end of the output needle 208 comes to bear against the proximal end 256 of the piston 210. Subsequent dispense of the first medicament 201 into the medicated module 202 then causes the piston 210 to move further distally in direction 248, thus causing the piston 210 to be fully pierced or ruptured by the piercing tip 254 of the output needle 208. When the piston 210 has been pierced, fluid communication is established between the output needle 208 and the engagement needle 206. Such fluid communication is shown in FIG. 3. Since fluid communication is achieved between the output needle 208 and the engagement needle 206, the first medicament 201 may be dispensed through the output 252 of output needle 208 into an injection site.

The predetermined amount of distal travel of the piston 210 that results in the piercing of the piston 210 preferably corresponds to when the piston 210 nears the bottom surface 228 of the medicated module 202 (and thus the point when all or substantially all of the second medicament has been forced into side hole 250). In other words, when the output needle 208 pierces the piston 210 and opens up fluid communication between the two needles, substantially all of the second medicament is preferably already dispensed from the medicated module 202. It should be understood that although preferably substantially all of the second medicament 224 is dispensed before the fluid communication between the two needles is opened up, it is not necessary that all or substantially all of the second medicament is dispensed from the medicated module. For example, in an embodiment, 80-95% of medicament 224 may be dispensed from the medicated module 202 before the fluid communication between the two needles is opened up. Other examples are possible as well. Further, it should be understood that some of the second medicament 224 may still be in needle 208 when the needle 208 pierces the piston 210. Still further, a small amount of the second medicament 224 may still be in the cavity 226 when the needle 208 pierces the piston 210.

After the user finishes dispensing of the first medicament 201, the user may remove the output needle 208 from the injection site. Then, the depleted medicated module 202 may be disposed of. Assuming that the drug delivery device 204 still holds some first medicament 201, the drug delivery device 204 may be reused by the patient as required.

In an embodiment, the medicated module may also include an air bleed feature 258. The purpose of this feature is to allow the pressure behind the larger diameter sliding seal to equalize as the piston 210 is forced distally during dispense, thereby helping to reduce the force that needs to be exerted by the primary medicament to drive the piston forward. In this preferred embodiment this air bleed feature has been shown as a simple side cored hole connecting the proximal region behind sealing feature 214 and atmospheric pressure, although other embodiments where such an air bleed feature are formed through non-air tight assembly of multiple components is possible. Further, if the volume of secondary medicament to be dispensed is small relative to the volume of air behind the sealing bead, or if an additional flexible element (preferably with relatively low strain rate) is incorporated into the region behind the seal, then a fully sealed solution may be practicable. Further, the first needle 206 and second needle 208 are preferably sealably fixed in the medicated module. For example, the needles can be sealably fixed in respective needle hubs. The retention cap 222 may serve as a needle hub for the first needle 206, as shown in FIGS. 2a and 3. Further, the second needle 208 may be fixed in needle hub 264.

A particular benefit of Applicants' proposed concept is the arrangement of the floating piston 210, which minimizes ullage of the first, primary medicament 201 in the medicated module 202 relative to the volume of the secondary medicament that it displaces during dispense. The piston 210 is arranged such that the cross-sectional area in contact with the second medicament 224 is substantially larger than the cross-sectional area of the floating piston 210 in contact with the first, primary medicament 201. Hence, to dispense the fixed volume of the second medicament 224, the axial displacement of the floating piston is minimal (i.e., substantially less than if the piston had only a single, common cross-sectional area). The ullage of the primary medicament is substantially the product of the cross-sectional area of the first portion and the swept displacement of the floating piston 210. As shown in FIG. 3, the swept displacement corresponds to the height H1 260. As the cross-sectional area in contact with the primary medicament 201 is small (relative to the cross-sectional area in contact with the fixed dose secondary medicament 224) and the swept displacement of the first and second portions of the floating piston is the same for both, the ullage of the primary medicament 201 in the medicated module after use is therefore less than the volume of the second medicament 224 dispensed from the module by a ratio equal to the ratio of the two respective cross-sectional areas of the floating piston.

In this example, the two cavities are cylindrical cavities. As discussed above, in this example, the radius of the second portion is three (3) times the radius of the first portion. Since A=πr2, the Asecond_portion=9Afirst_portion or ( 1/9)Asecond_portion=Afirst_portion. Accordingly, the ullage of the primary medicament 201 in the medicated module after use is therefore 1/9th of the volume of the second medicament 224 dispensed from the module. Specifically, since the volume of cylinder is V=πr2h=Ah and rsecond_portion=3rfirst_portion: (i) Vfirst_portion=π(rfirst_portion)2h; and (ii) Vsecond_portion=π(3rfirst_portion)2h=9π(rfirst_portion)2h=9Vfirst_portion. Accordingly, the original volume of the second medicament 224 in cavity 226 is nine (9) times the ullage volume of the first medicament 201 in cavity 242.

Other examples are possible as well. For instance, if the radius of the second portion is four (4) times the radius is the first portion, the ullage of the primary medicament 201 in the medicated module after use would therefore be 1/16th of the volume of the second medicament 224 actually dispensed. In these above examples, it is assumed that the cavities are cylindrical. However, it should be understood that the cavities could be other shapes, such as cubical, rectangular, or other polygon-based shapes.

In the example of FIGS. 2-3, the height of indentation 225 is preferably slightly greater than or equal to the height of original cavity 226 of the second medicament 224. Therefore, when the piston 210 travels a distance corresponding to H1 260, a part of the first portion 212 will remain in sliding, fluid tight engagement with the indentation, thereby preventing medicament 201 from flowing beyond the first portion 212.

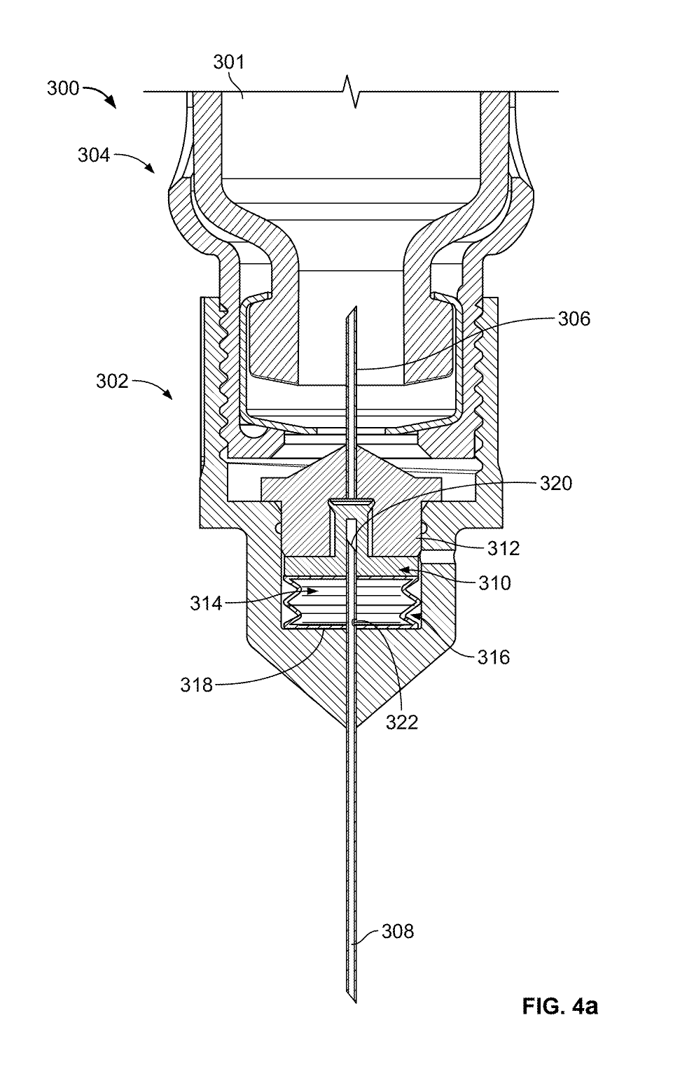

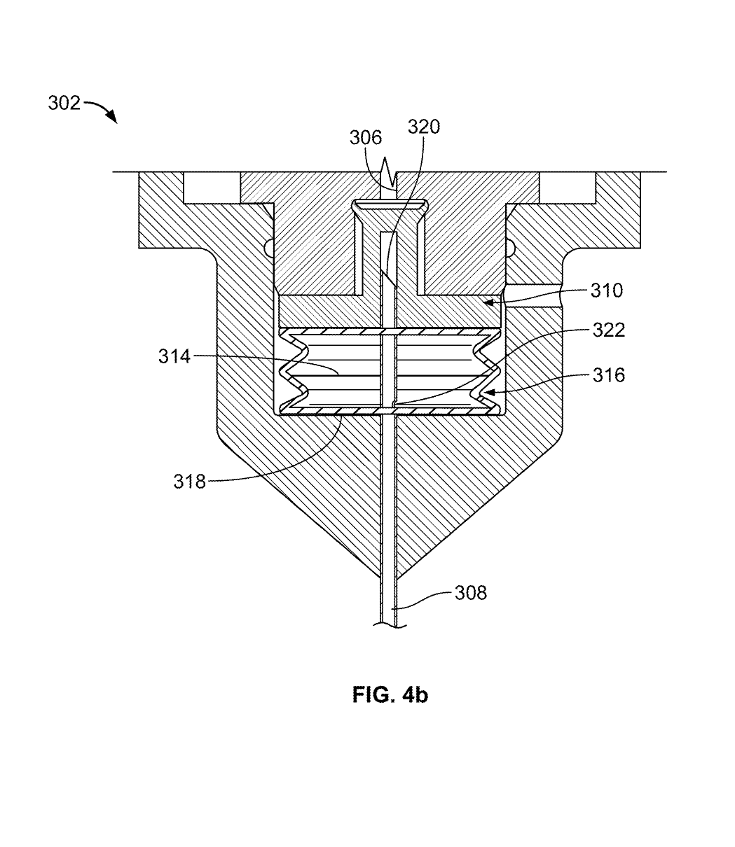

In an alternative embodiment of Applicants' proposed concept, the second medicament is contained between a flexible membrane and the lower surface of the medicated module. Such an alternative embodiment is depicted in FIGS. 4a and 4b. This medicated module is similar in many respects to the medicated module 202 shown in FIGS. 2-3, and thus is not described in as great of detail.

Specifically, FIGS. 4a-b illustrate a drug delivery system 300 including medicated module 302 and primary drug delivery device 304 that contains first primary medicament 301. The medicated module 302 includes a first needle 306 (i.e., engagement needle) and a second needle 308 (i.e., output needle). The medicated module 302 also includes floating, dual cross-section piston 310, medicament retention cap 312, and second medicament 314. Unlike the example of FIGS. 2-3, the second medicament is contained in a flexible membrane 316.

The flexible membrane 316 can be pre-filled with the second medicament and then dropped into a needle-sub-assembly during the manufacturing process. During dispense, at a predetermined axial displacement (e.g., just before or when the second portion reaches a distal surface such as bottom surface 318), the piercing tip 320 of the output needle pierces the floating piston 310 so that fluid communication between the engagement needle 306 and output needle 308 is achieved. As this occurs, the floating piston 310 prevents the primary medicament 301 from filling the flexible membrane 316 by substantially occluding the side hole 322 present in the output needle 308. As shown in the figures, the flexible membrane will be pierced (both top and bottom surfaces) by the output needle 308 during assembly in the manufacturing facility. During dispense the secondary medicament is forced into and out of the needle (located between the upper and lower membranes) as described in the embodiment above under the action of the piston moving distally. Essentially the membrane is simply replacing the need for the lower seal on the floating piston and also may make filling and assembly easier. The flexible membrane can be manufactured separately and then dropped into the needle sub-assemblies, rather than the medicament having to be filled directly into the sub-assemblies.

An exemplary benefit of containing the secondary medicament in a flexible membrane compared to the first embodiment is improved sealing of the second medicament due to the ease of the flexible membrane to provide a static fluid seal compared to the dynamic seal required of the floating piston in the first embodiment.

In yet another embodiment of Applicants' proposed concept, the floating piston may be present in the output needle of a medicated module. This embodiment is similar in many respects to the previously discussed alternative embodiments, and thus will not be described in as much detail. However, the piston of this alternative embodiment is not a dual cross-section piston and the piston is arranged within the output needle, preferably near the outlet tip. This embodiment is preferably used with a low volume, high concentration secondary medicament. For example, this embodiment is preferably used with a second medicament having a volume less than 0.5 micro liters.

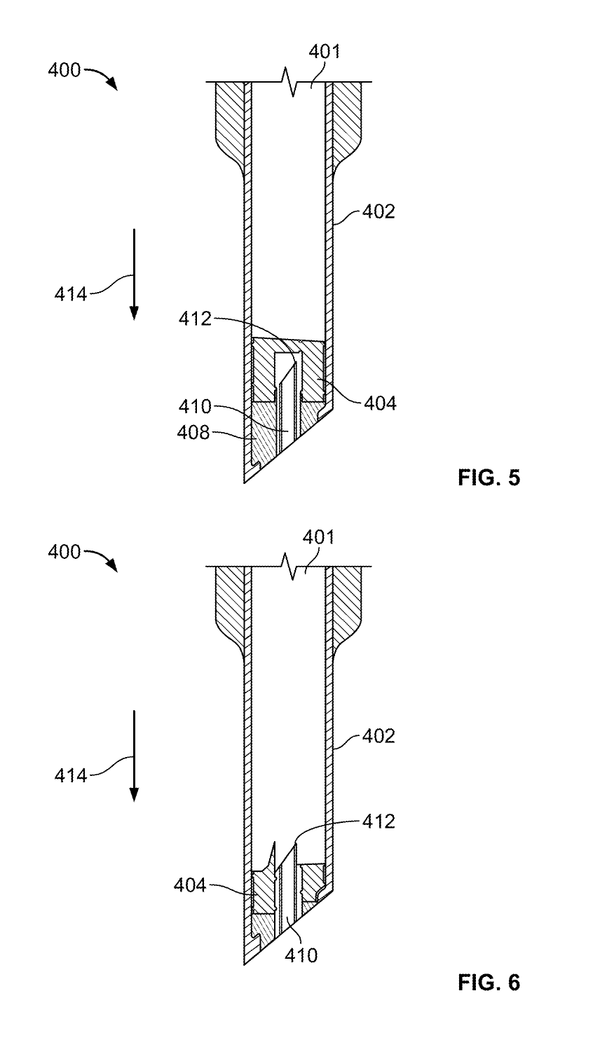

FIGS. 5 and 6 depict partial cross-sectional views of an exemplary medicated module 400. Specifically, FIG. 5 depicts a cross-section through an output needle of the third embodiment prior to dispense, and FIG. 6 depicts a cross-section of the output needle during dispense of the primary medicament.

After the medicated module 400 is attached to a drug delivery device holding a primary medicament 401, the output needle 402 holds the first medicament 401, a floating piston 404, a second medicament 408, and a piercing needle 410 with piercing tip 412. Before dosing, the floating piston 404 separates the first medicament 401 from the second medicament 408. When a user dispenses the medicament, the first medicament forces the floating piston 404 in distal direction 414. This action thereby forces preferably substantially all of the second medicament 408 out the output needle 402. After a predetermined amount of displacement of the floating piston 404, the piercing tip 412 pierces the floating piston 404, as shown in FIG. 6. After the floating piston is pierced, the first medicament may then be dispensed from the output needle 402 via the piercing needle 410. The piercing needle may be held in place with the output needle by a side rib to connect to the inner wall of the output needle, most preferably using three ribs.

Applicants' proposed concept also includes a method for dispensing a non-user settable dose of one medicament followed by a user-settable dose of a primary medicament. The method includes the step of attaching a medicated module to a drug delivery device, such as the medicated modules and drug delivery devices illustrated in FIGS. 1-6. As described above, the drug delivery device has a primary drug reservoir holding the primary medicament. Further, the medicated module includes a first needle, a second needle, a dual cross-section, floating piston, and a second medicament. The second medicament is located on a distal side of the piston in a cavity defined by a distal surface of the medicated module and a distal end of the second portion of the piston. After the medicated module is attached to the drug delivery device, (i) the first needle is in fluid communication with the drug reservoir and (ii) the piston prevents fluid communication between the first and second needle. The method further includes the step of setting a dose of the primary medicament contained in the primary drug reservoir using a single dose setter of the drug delivery device.

The method then includes activating a dose button on the drug delivery device to cause the set dose of the primary medicament from the primary drug reservoir to flow in the distal direction toward the piston. During dispensing, the first medicament flows through the first needle into a second cavity formed by the retention feature and the first portion of the piston. The first medicament forces the piston to move in a distal direction toward the distal surface, and the second portion of the piston forces substantially all of the second medicament to flow through the second needle. After substantially all of the second medicament is forced to flow through the second needle, the second needle fully pierces the piston, thereby opening up fluid communication between the first needle and the second needle. The method then includes the step of forcing the primary medicament to flow through the second needle.

As described above, Applicants' proposed concept beneficially allows for sequential dosing of a secondary medicament stored in a medicated module followed by a primary medicament from a primary drug delivery device. The proposed dual cross-section, floating piston ensures that the primary medicament is only delivered after the secondary medicament is substantially dispensed. Thus, beneficially, the proposed concept prevents or limits the mixing of the two medicaments within the device. This could be beneficial for applications whereby the efficacy of the combined medication reduces over time and so it is best for the mixing to occur as close to in vivo as possible to maximum the therapeutic effect.

Further, as mentioned above, it may be preferable for the ullage volume in the medicated module (post dispense) to be smaller than the volume of the fixed dose secondary medicament dispensed by it. This is especially the case where the volume of the fixed dose secondary medicament needs to be relatively large, relative to the potential dose from the multi-use or single use drug delivery device having the primary medicament (e.g., greater 0.05 milliliters (ml) in the example of a pen-type injection device for the delivery of insulin). This proposed arrangement is beneficial for ullage concerns for a number of reasons. For instance, this arrangement reduces the amount of medicament that is wasted by being left in the medicated module following use and reduces the degree to which the dialed dose (i.e., what is indicated to the user) on the drug delivery device needs to be offset to accommodate the effect of ullage in the medicated module. The arrangement has the benefit of sequentially dosing the medicament so that the two medicaments have minimum opportunity for mixing ex-vivo, and also minimizing the amount of ullage left in the medicated module post use (relative to the volume of the fixed dose secondary medicament dispensed).

The connection or attachment between the medicated module of the above descried embodiments may contain additional features (not shown), such as connectors, stops, splines, ribs, grooves, and the like design features, that ensure that specific medicated module are attachable only to matching drug delivery devices. Such additional features would prevent the insertion of a non-appropriate medicated module to a non-matching injection device.

The shape of the medicated module may be a cylindrical body or any other geometric shape suitable for defining a fluid reservoir or for containing discrete self-contained reservoir of the medicament in the medicated module and for attaching one or more needle cannula. The medicated module can be manufactured from any drug contact suitable material. The integrated output needle can be any needle cannula suitable for subcutaneous or intramuscular injection. Preferably the medicated module is provided by a drug manufacturer as a stand-alone and separate device that is sealed to preserve sterility. The sterile seal of the module is preferably designed to be opened automatically, e.g. by cutting, tearing or peeling, when the medicated module is advanced or attached to the drug delivery device by the user.

The medicated module of Applicants' concept should be designed to operate in conjunction with a multiple use injection device, preferably a pen-type multi-dose injection device, similar to what is illustrated in FIG. 1. The injection device could be a reusable or disposable device. By disposable device it is meant an injection device that is obtained from the manufacturer preloaded with medicament and cannot be reloaded with new medicament after the initial medicament is exhausted. The device may be a fixed dose or a settable dose and preferably a multi-dose device, however, in some cases it may be beneficial to use a single dose, disposable device.

A typical drug delivery device contains a cartridge or other reservoir of medication. This cartridge is typically cylindrical in shape and is usually manufactured in glass. The cartridge is sealed at one end with a rubber bung and at the other end by a rubber septum. The drug delivery pen is designed to deliver multiple injections. The delivery mechanism is typically powered by a manual action of the user, however, the injection mechanism may also be powered by other means such as a spring, compressed gas or electrical energy.

Exemplary embodiments of the present invention have been described. Those skilled in the art will understand, however, that changes and modifications may be made to these embodiments without departing from the true scope and spirit of the present invention, which is defined by the claims.

1. A medicated module attachable to a drug delivery device, the drug delivery device having a drug reservoir holding a first medicament, the medicated module comprising:
a first needle;
a second needle;
a piston, wherein the piston has a first portion and a second portion, wherein an area of a cross-section of the first portion is smaller than an area of a cross-section of the second portion; and
a retention feature having an indentation, wherein at least part of the first portion of the piston is located in the indentation, and wherein the second portion of the piston is located on a distal side of the retention feature;
a second medicament, wherein the second medicament is located on a distal side of the piston in a cavity, wherein the cavity is defined by a distal surface of the medicated module and a distal end of the second portion of the piston,
wherein, after the medicated module is attached to the drug delivery device, (i) the first needle is in fluid communication with the drug reservoir and (ii) the piston prevents fluid communication between the first and second needle,
wherein, during dispensing, the first medicament flows through the first needle into a second cavity formed by the retention feature and the first portion of the piston, and wherein the first medicament forces the piston to move in a distal direction toward the distal surface of the medicated module, wherein the second portion of the piston forces substantially all of the second medicament to flow through the second needle, and
wherein, after substantially all of the second medicament is forced to flow through the second needle, the second needle fully pierces the piston, thereby opening up fluid communication between the first needle and the second needle.

2. The medicated module (of claim 1, wherein after fluid communication is opened up between the first needle and the second needle, a dose of the first medicament is forced to flow from the second cavity through the second needle.

3. The medicated module of claim 1, wherein the area of the cross-section of the second portion of the piston is at least double the area of the cross-section of the first portion of the piston.

4. The medicated module of claim 1, wherein the second portion of the piston is in sliding, fluid-tight engagement with an inner wall of the medicated module.

5. The medicated module of claim 1, wherein, when the piston contacts the distal surface, a part of the first portion of the piston remains in the indentation.

6. The medicated module of claim 1, wherein the first portion of the piston is in sliding fluid-tight engagement with an inner wall of the indentation.

7. The medicated module of claim 1, wherein the second needle comprises a side hole for receiving the second medicament.

8. The medicated module of claim 1, wherein the medicated module further comprises an air-bleed feature.

9. The medicated module of claim 1, wherein the first needle is fixed in a first needle hub that is a portion the retention feature.

10. The medicated module of claim 1, wherein after a dose of the first medicament is dispensed from the medicated module, an ullage of the first medicament remaining in the second cavity is less than half of the volume of the second medicament that was initially in the cavity prior to dispense.

11. A medicated module attachable to a drug delivery device, the drug delivery device having a drug reservoir holding a first medicament, the medicated module comprising:
a first needle;
a second needle;
a piston, wherein the piston has a first portion and a second portion, wherein an area of a cross-section of the first portion is smaller than an area of a cross-section of the second portion; and
a retention feature having an indentation, wherein at least part of the first portion of the piston is located in the indentation, and wherein the second portion is located on a distal side of the retention feature; and
a second medicament, wherein the second medicament is located in a flexible membrane on a distal side of the piston,
wherein, after the medicated module is attached to the drug delivery device, (i) the first needle is in fluid communication with the drug reservoir and (ii) the piston prevents fluid communication between the first and second needle,
wherein, during dispensing, the first medicament flows through the first needle into a cavity formed by the retention feature and the first portion of the piston, and wherein the first medicament forces the piston to move in a distal direction toward a distal surface of the medicated module, wherein the second portion of the piston forces substantially all of the second medicament to flow through the second needle, and
wherein, after substantially all of the second medicament is forced to flow through the second needle, the second needle fully pierces the piston, thereby opening up fluid communication between the first needle and the second needle.

12. The medicated module of claim 11, wherein the area of the effective cross-section of the membrane containing the second medicament is at least double the area of the cross-section of the first portion of the piston.

13. A medicated module attachable to a drug delivery device, the drug delivery device having a drug reservoir holding a first medicament, the medicated module comprising:
an output needle containing a second medicament;
a floating piston disposed in the output needle;
a piercing needle having a piercing tip, wherein the piercing needle is securely fixed in the output needle,
wherein dispense of the first medicament forces the floating piston to move in a distal direction, wherein the distal movement forces the second medicament out of the output needle, and wherein, at a predefined amount of distal movement in the distal direction, the piercing tip fully pierces the floating piston, thereby opening up fluid communication between the first medicament and an output of the output needle.

14. The medicated module of claim 13, wherein a volume of the second medicament is less than 0.5 micro liters.
